(19) United States
(12) Patent Application Publication
(10) Pub. No.: US 2024/0415930 A1
(43) Pub. Date: Dec. 19, 2024

(54) COMPOSITIONS AND METHODS FOR ENHANCING BRAIN PERFUSION

(71) Applicant: Washington University, St. Louis, MO (US)

(72) Inventors: Jonathan KIPNIS, St. Louis, MO (US); Antoine DRIEU, St. Louis, MO (US)

(73) Assignee: Washington University, St. Louis, MO (US)

(21) Appl. No.: 18/682,707

(22) PCT Filed: Aug. 4, 2022
(86) PCT No.: PCT/US2022/039418

§ 371 Date: Feb. 9, 2024

Related U.S. Application Data
(60) Provisional application No. 63/229,400, filed on Aug. 4, 2021.
(60) Provisional application No. 63/229,507, filed on Aug. 5, 2021.

(51) Int. Cl. A61K 38/19; A61P 25/28

(52) U.S. Cl. CPC A61K 38/193; A61P 25/28

(57) ABSTRACT

Compositions and methods for the treatment, prevention, or reversal of a brain disease or disorder characterized by accumulation of debris within a brain in a subject in need by administration of a therapeutically effective amount of a composition comprising a perivascular macrophage (PVM) stimulating compound are disclosed.

### CROSS-REFERENCE TO RELATED APPLICATIONS

This application claims priority from U.S. Provisional Application Ser. No. 63/229,400 filed on Aug. 4, 2021, the content of which is incorporated herein by reference in its entirety. This application also claims priority from U.S. Provisional Application Ser. No. 63/229,507 filed on Aug. 5, 2021, the content of which is incorporated herein by reference in its entirety.

### STATEMENT REGARDING FEDERALLY SPONSORED RESEARCH OR DEVELOPMENT

Not applicable.

### MATERIAL INCORPORATED-BY-REFERENCE

Not applicable.

### FIELD OF THE DISCLOSURE

The present disclosure generally relates to compositions and methods for enhancing brain perfusion.

### BACKGROUND OF THE DISCLOSURE

Brain border-associated macrophages are strategically located to protect the brain from pathogens, as well as sampling brain antigens. Perivascular macrophages (PVMS) are long-lived cells residing in the cerebrospinal fluid (CSF) and more precisely in the perivascular space (PVS), a physical space delineated by the endothelium and the astrocytic endfeet. Recent studies demonstrated that the CSF could diffuse from the PVS to the brain parenchyma. The CSF will then mix with the local interstitial fluid and finally exit the brain, carrying along the brain's debris.

### SUMMARY OF THE INVENTION

Among the various aspects of the present disclosure is the provision of compositions and methods for enhancing brain perfusion.

Briefly, therefore, the present disclosure is directed to methods and compositions to treat, prevent, or reverse a brain disorder.

The present teachings include compositions for a perivascular macrophage (PVM) stimulating compound.

In one embodiment, a method of treating, preventing, or reversing a brain disease or disorder characterized by accumulation of debris within a brain in a subject in need is disclosed that includes administering a therapeutically effective amount of a composition comprising a perivascular macrophage (PVM) stimulating compound. In some aspects, the PVM stimulating compound includes macrophage colony stimulating factor (M-CSF). In some aspects, the brain disease or disorder characterized by the accumulation of debris within the brain in a subject is selected from Alzheimer's Disease (AD) and Parkinson's Disease (PD). In some aspects, the composition is administered by intracisternal magna (ICM) injection.

Other objects and features will be in part apparent and in part pointed out hereinafter.

### DESCRIPTION OF THE DRAWINGS

The following drawings illustrate various aspects of the disclosure.

FIG. 1A is a set of two images of a whole brain section showing that PVMs are located in leptomeninges and large blood vessels.

FIG. 1B is a pair of images illustrating that PVMs and microglia can be stained with Iba1 antibody, but PVMs can be distinguished by their location and the specific expression of CD206.

FIG. 1C is a pair of images illustrating that PVMs can be distinguished from microglia by Lyve1 staining.

FIG. 1D is a schematic diagram summarizing an experimental design of PVM/microglia depletion using PLX 56222, combined with GFP-skull transplantation.

FIG. 1E is a set of three images of whole brain showing GFP cells from the GFP-transplanted skull sitting on top of the brain.

FIG. 1F is a set of images illustrating that the GFP positive cells found in the brain were exclusively CD206 positive, and located on both leptomeninges and perivascular space. Any of the Iba1 positive/CD206 negative cells located in the brain parenchyma (i.e., microglial cells) were GFP positive.

FIG. 2A is a schematic diagram illustrating an experimental design to study CSF flow dynamics, where tracers (here, Ovalbumine, 45 kDa) were injected into the cisterna magna (ICM). After injection, tissues were harvested for histological analyses.

FIG. 2B is a set of images showing that tracers were up taken by PVMs (OVA positive/CD206 positive cells).

FIG. 2C is a schematic diagram summarizing an experimental design of specific PVM depletion using ICM injection of PBS- or clodronate-encapsulated liposomes (CLO).

FIG. 2D is a set of images obtained after one week of PBS- or CLO-liposome injection, in which PVM depletion was evaluated by counting the number of CD206 cells.

FIG. 2E is a graph summarizing the quantification of CD206 cell percentages in PBS- and CLO-treated mice (% vs PBS group; 80% depletion).

FIG. 2F is a schematic diagram summarizing an experimental design wherein OVA was injected ICM one week after PVM depletion to evaluate CSF flow.

FIG. 2G is a set of representative whole brain images showing OVA coverage one hour after ICM injection. Higher magnification pictures were obtained at the middle cerebral artery (MCA) level, where most of the tracer was found.

FIG. 2H is a graph summarizing the quantification of OVA coverage at the MCA level for PBS- and CLO-treated mice.

FIG. 2I is a representative coronal brain section showing OVA coverage in PBS- and CLO-treated mice.

FIG. 2J is a graph summarizing the quantification of OVA coverage on coronal sections in PBS- and CLO-treated mice.

FIG. 2K is a schematic diagram summarizing an experimental design of the evaluation of CSF flow dynamics in vivo.

FIG. 2L is a set of representative images showing OVA coverage at the MCA level in vivo over one hour in PBS- and CLO-treated mice.

FIG. 2M is a graph summarizing the quantification of OVA flow dynamics over an hour in PBS- and CLO-treated mice.

FIG. 2N is a schematic diagram summarizing an experimental design of photoacoustic imaging of the cerebro-vasculature in vivo. Ten percent of CO₂ in the nose cone was able to create robust vasodilation.

FIG. 2O is a set of representative images showing arteries before and during CO₂ challenges in PBS- and CLO-treated mice.

FIG. 2P is a graph summarizing the quantification of arterial diameter fold increase after the CO₂ challenges in PBS- and CLO-treated mice.

FIG. 2Q is a schematic diagram summarizing an experimental design to determine the effect of vasomotion induced by systemic dobutamine injection after PVM depletion on CSF flow.

FIG. 2R is a set of representative whole brain and MCA images showing OVA coverages in PBS- and CLO-treated mice.

FIG. 2S is a second set of representative whole brain and MCA images showing OVA coverage in PBS- and CLO-treated mice.

FIG. 2T is a graph summarizing the quantification of OVA coverage at the MCA level in PBS- and CLO-treated mice.

FIG. 2U is a set of representative coronal brain sections showing OVA coverages in PBS- and CLO-treated mice.

FIG. 2V is a graph summarizing the quantification of OVA coverages on coronal sections in PBS- and CLO-treated mice.

FIG. 3A is a map of single cell RNA-Sequencing of the whole brain.

FIG. 3B is a map showing six clusters of PVMs identified in the dataset.

FIG. 3C is a map of RNA Magnet plugin that demonstrated that PVM clusters 0 and 1 interacted with pericytes, while PVM cluster 2 mainly interacted with vascular smooth muscle cells (VSMC) mainly located at the arterial level.

FIG. 3D is a graph summarizing RNA data results that demonstrates that PVM cluster 2 could be distinguished from clusters 0-1 by its specific expression of scavenger markers such as Cd163 and Lyve1.

FIG. 3E is a set of representative images showing the locations of Lyve1 positive PVMs at the arterial level (stained for alpha Smooth Muscle Actin, aSMA).

FIG. 3F is a higher magnification of one image from FIG. 3E showing potential interaction between a Lyve1 positive PVM and a smooth muscle cell.

FIG. 3G is a graph summarizing the quantification of CD206 positive PVMs on aSMA positive arteries and aSMA negative blood vessels.

FIG. 3H is a graph summarizing the quantification of Lyve1 positive PVMs on aSMA positive arteries and aSMA negative blood vessels.

FIG. 3I is a schematic diagram summarizing an experimental design to determine the effect of CSF flow on Lyve1-Cre/Csf1r-Flox mice. These mice lack specifically Lyve1 positive PVMs.

FIG. 3J is a set of representative images showing CD206 staining on Lyve1-Cre/Csf1r-Flox mice.

FIG. 3K is a graph summarizing the quantification of PVM depletion in the 2 mouse models described in FIG. 3I.

FIG. 3L is a set of representative whole brain and MCA images showing OVA coverage in the mouse models described in FIG. 3I.

FIG. 3M is a graph summarizing the quantification of OVA coverage at the MCA level of the two mouse models described in FIG. 3I.

FIG. 3N is a set of images of representative coronal brain sections showing OVA coverage in the mouse models described in FIG. 3I.

FIG. 3O is a graph summarizing the quantification of OVA coverage on coronal sections in the mouse models described in FIG. 3I

FIG. 3P is a set of representative stainings of Lyve1 and MHC-II in young adult (3-month-old) and aged (24-month-old) mice. Lyve1 and MHC-II were mainly colocalized with CD206 positive PVMs.

FIG. 3Q is another set of representative stainings of Lyve1 and MHC-II in young adult (3-month-old) and aged (24-month-old) mice. Lyve1 and MHC-II were mainly colocalized with CD206 positive PVMs.

FIG. 3R is a graph summarizing the quantification of PVMs in 3-month-old and 24-month-old mice.

FIG. 3S is a graph summarizing the quantification of Lyve1 positive PVMs in 3-month-old and 24-month-old mice.

FIG. 3T graph summarizing the quantification of MHC-II positive PVMs in 3-month-old and 24-month-old mice.

FIG. 3U is a schematic diagram summarizing an experimental design to determine the effect of M-CSF treatment on CSF flow in aged mice.

FIG. 3V is a set of representative whole brain and MCA images showing OVA coverage in M-CSF- and aCSF-treated mice.

FIG. 3W is a graph summarizing the quantification of OVA coverage at the MCA level of M-CSF- and aCSF-treated mice.

FIG. 3X is a set of representative coronal brain sections showing OVA coverage in M-CSF- and aCSF-treated mice.

FIG. 3Y is a graph summarizing the quantification of OVA coverage on coronal sections from M-CSF- and aCSF-treated mice.

FIG. 4A is a schematic diagram summarizing an experimental design wherein mice received an intracisternal magna (ICM) injection of PBS- or clodronate-liposomes. Mice receiving clodronate liposomes (CLO group) showed 85% PVM depletion one week after only one clodronate injection. Mice then received an ICM injection of different fluorescent tracers (OVA; 45 kDa and FITC-dextran;4 kDa). One hour after ICM injection of the fluorescent tracers, brains were harvested to analyze tracer diffusion.

FIG. 4B is a set of representative brain images from the mice from the experiment described in FIG. 4A. PVM depleted mice showed impaired tracer diffusion/coverage

FIG. 4C is a graph summarizing the quantification of OVA coverage in PBS- and CLO-treated mice. PVM depleted mice showed impaired tracer diffusion/coverage

FIG. 4D is a set of representative brain images of PBS- and CLO-treated mice. PVM depleted mice showed impaired tracer diffusion/coverage.

FIG. 4E is a graph summarizing the quantification of FITC-Dextran coverage in the brains of PBS- and CLO-treated mice.

FIG. 5A is a schematic diagram summarizing an experimental design wherein mice received an ICM injection of IgG (control) or an anti-CSF1R antibody. One week later, OVA was administered via ICM injection, and tissues were harvested one hour post-injection.

FIG. 5B is a set of representative brain images of IgG and anti-CSF1R treated mice.

FIG. 5C is a graph summarizing the quantification of OVA coverage in IgG and anti-CSF1R treated mice. OVA diffusion/coverage was impaired in CSF1R-treated mice, even though the number of PVMs was equivalent between groups.

FIG. 5D is a set of representative brain images of IgG and anti-CSF1R treated mice.

FIG. 5E is a graph summarizing the quantification of CD206 cell density from the images described in FIG. 5D.

FIG. 5F is a set of images from the brains of IgG and anti-CSF1R treated mice. PVMs were unable to uptake the tracer and downregulated scavenger molecules such as Lyve1.

FIG. 5G is a graph summarizing the quantification of OVA+ cells in IgG and CSF1R treated mice.

FIG. 5H is a graph summarizing the quantification of Lyve1+ cells in IgG and CSF1R treated mice.

FIG. 6A is a schematic diagram summarizing an experimental design wherein two-month-old 5×fAD mice received two IgG (control) or anti-CSF1R antibody injections (two-weeks interval between injections). Brains were harvested a month after the first injection and stained for AB to evaluate plaque loads.

FIG. 6B is a set of representative brain images of IgG and anti-CSF1R treated mice. Mice receiving the anti-CSF1R antibody showed more AB accumulation than IgG-treated mice.

FIG. 6C is a graph summarizing the quantification of AB coverage in the brains of IgG and anti-CSF1R treated mice.

FIG. 7A is a schematic diagram summarizing an experimental design wherein three-month-old and 24-month-old mice received OVA ICM to check tracer diffusion into the brain. Brains were harvested one hour after tracer diffusion.

FIG. 7B is a set of representative brain images of 3- and 24-month-old mice from the experiment described in FIG. 7A.

FIG. 7C is a graph summarizing the quantification of OVA coverage percent in the mice from the experiment described in FIG. 7A. Old mice had impaired OVA diffusion into the brain relative to young mice.

FIG. 7D is a schematic diagram summarizing an experimental design wherein old mice were treated with M-CSF (or aCSF as a control group). Six hours after treatment, mice received OVA via ICM injection, and brains were harvested one hour after OVA diffusion.

FIG. 7E is a set of representative images from M-CSF and aCSF-treated mice.

FIG. 7F is a graph summarizing the quantification of OVA coverage in M-CSF and aCSF treated mice. Mice receiving M-CSF showed an increase in OVA diffusion/coverage compared to aCSF-treated mice.

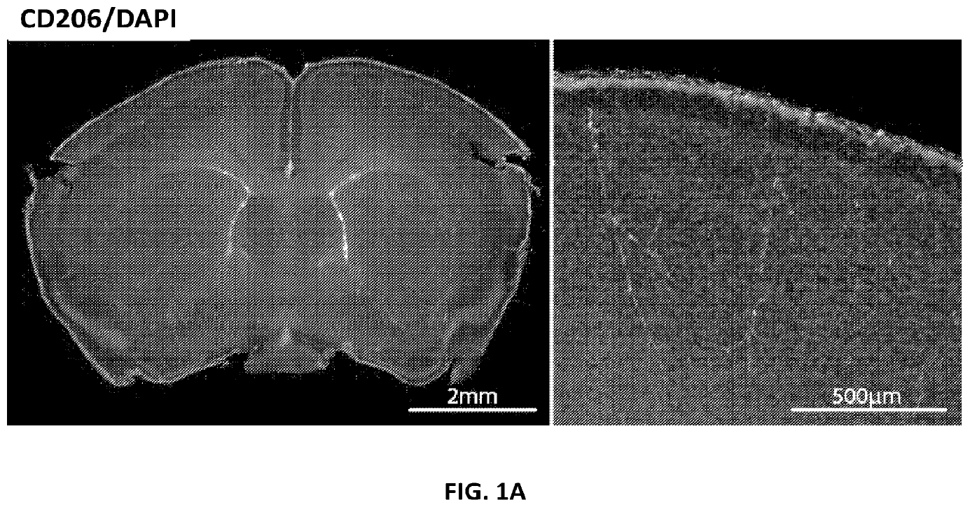

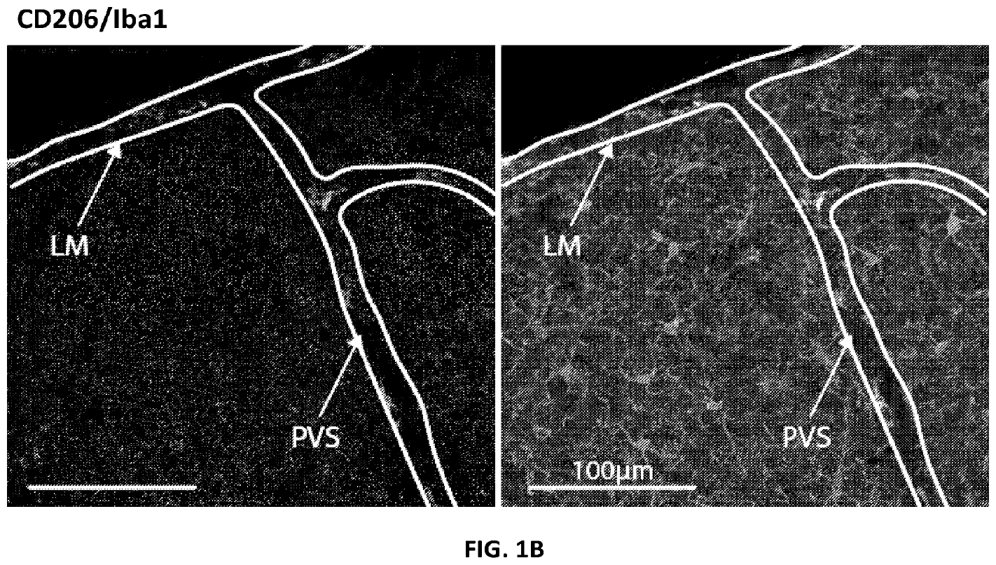

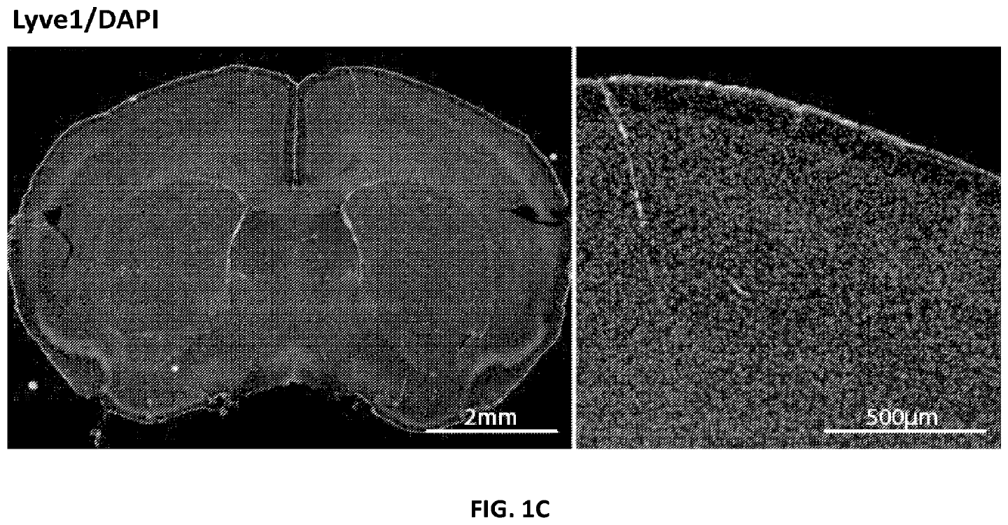

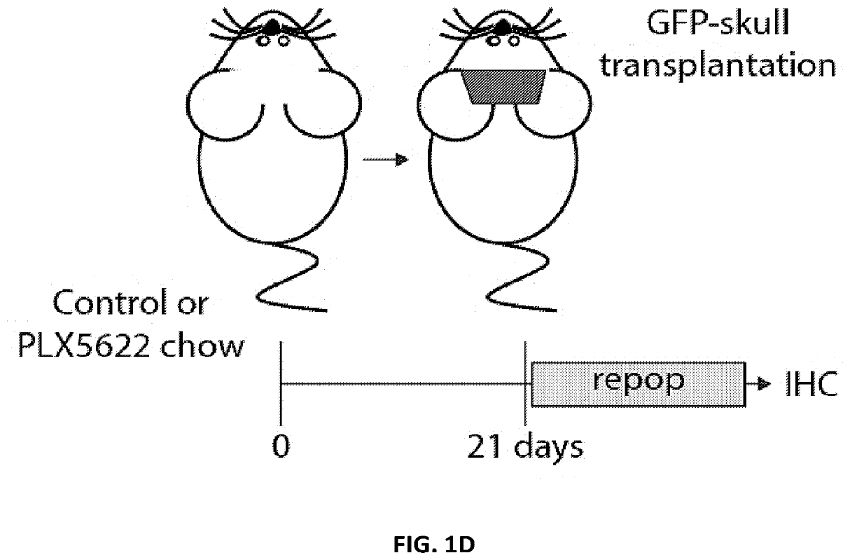

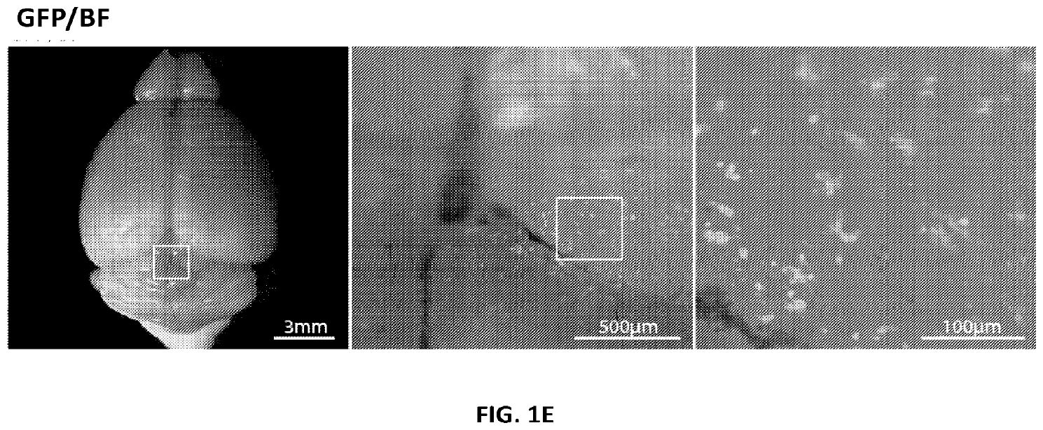

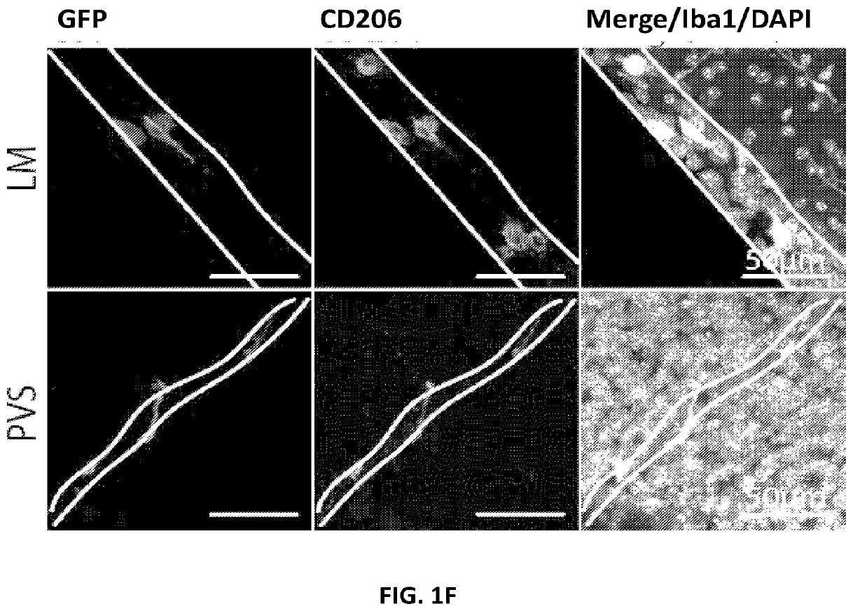

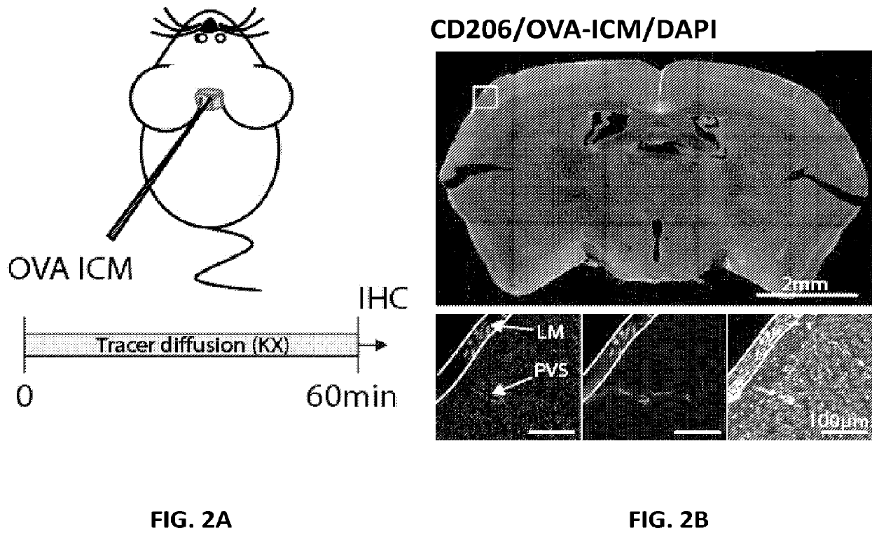

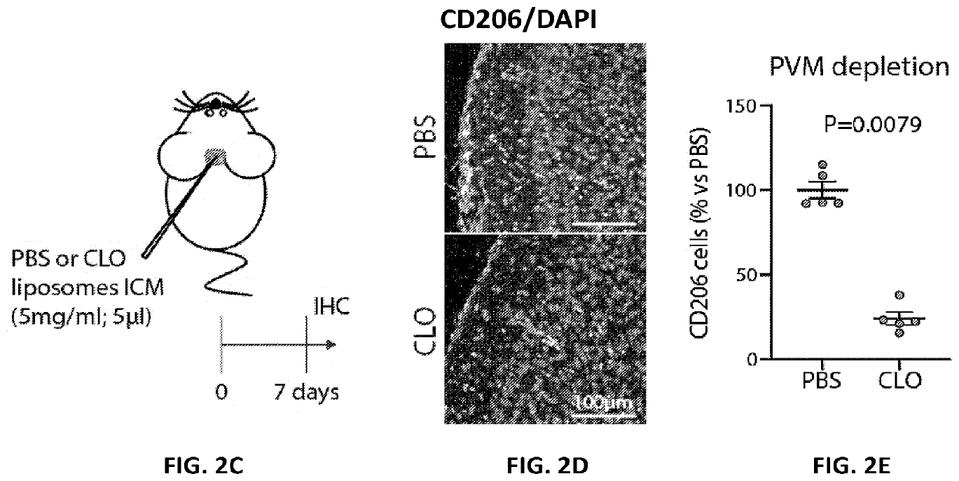

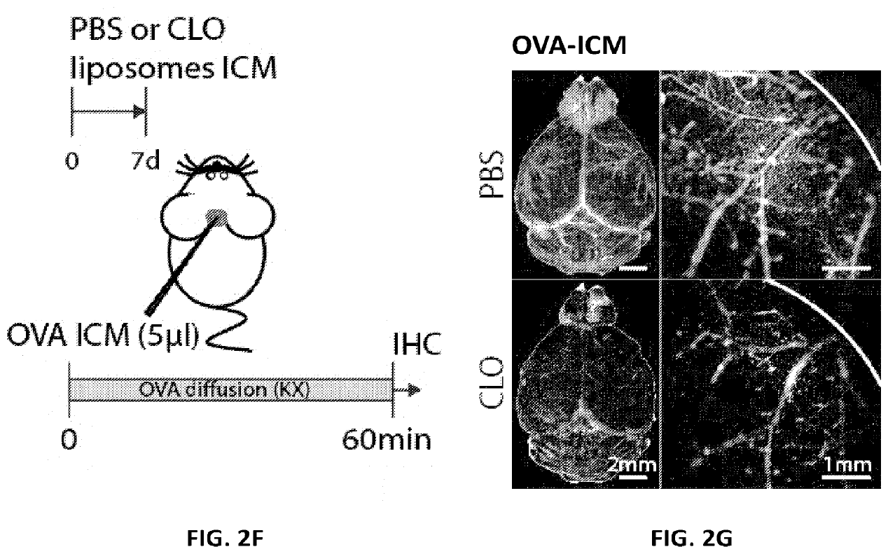

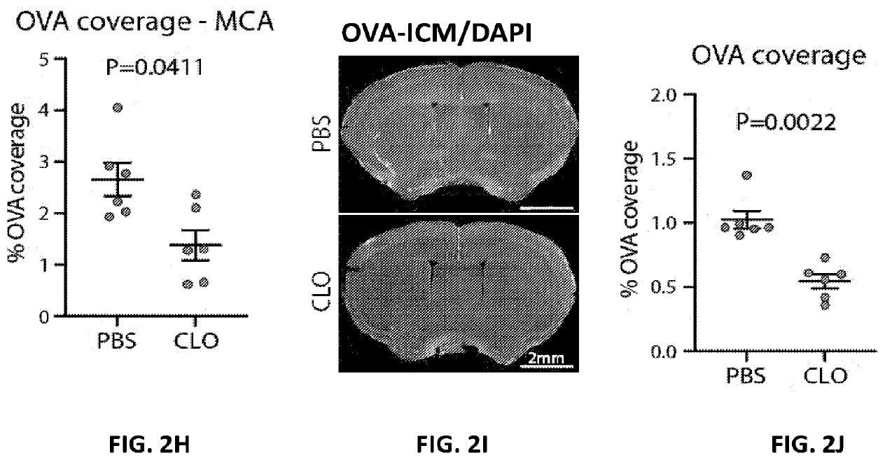

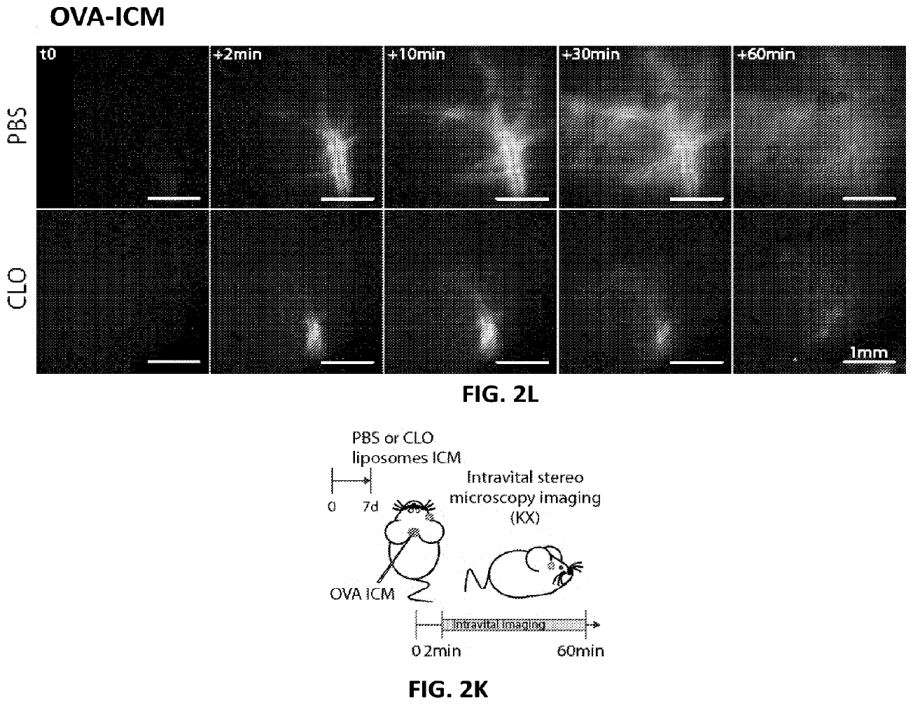

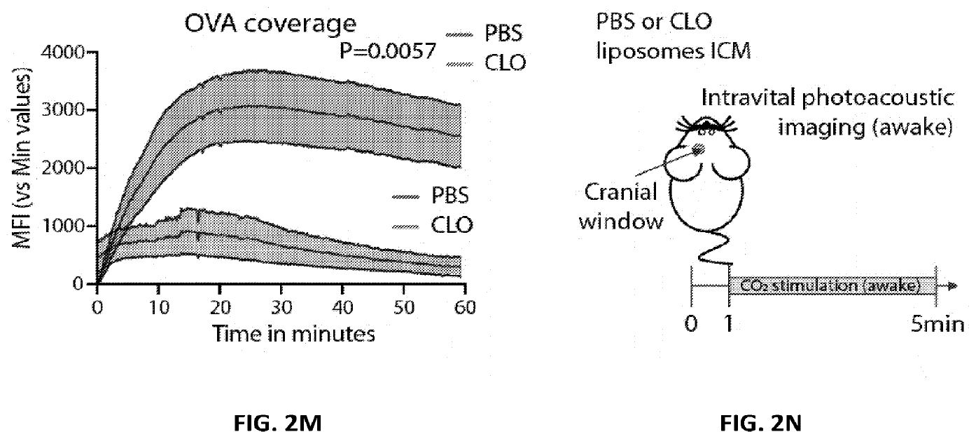

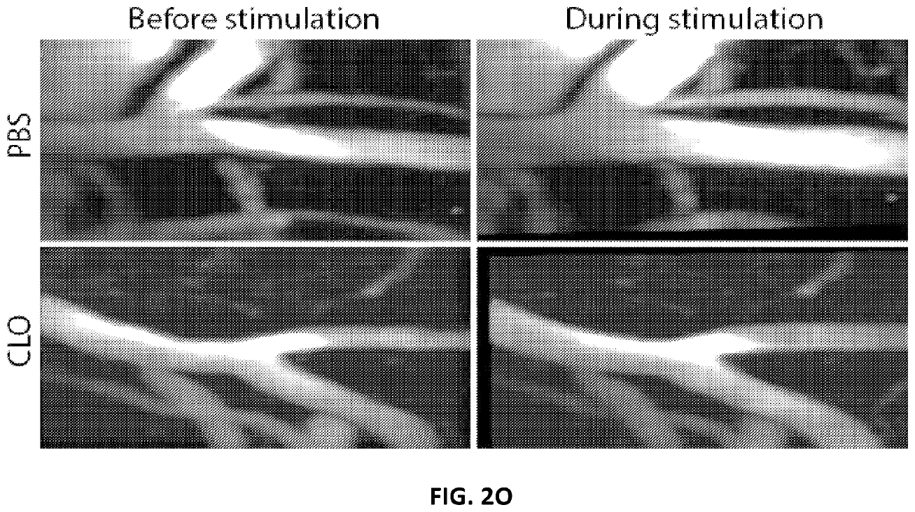

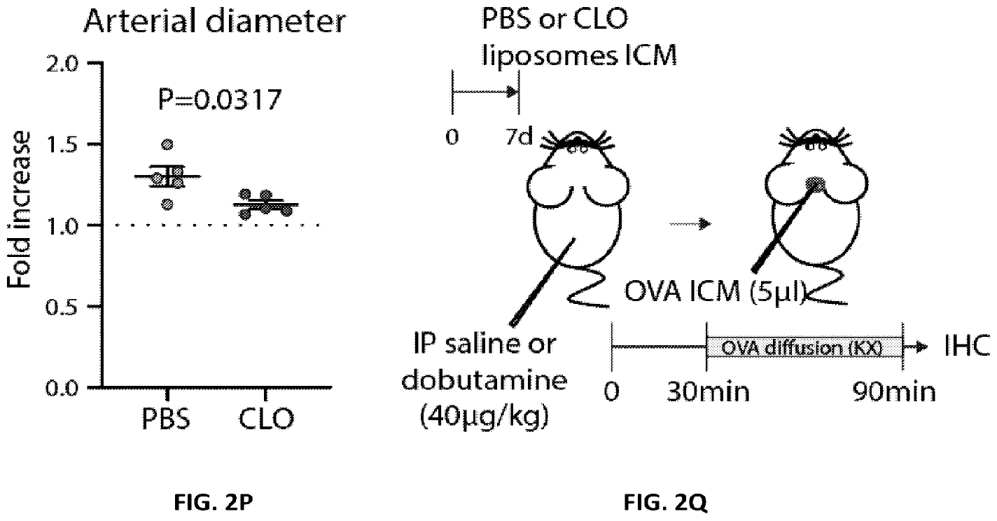

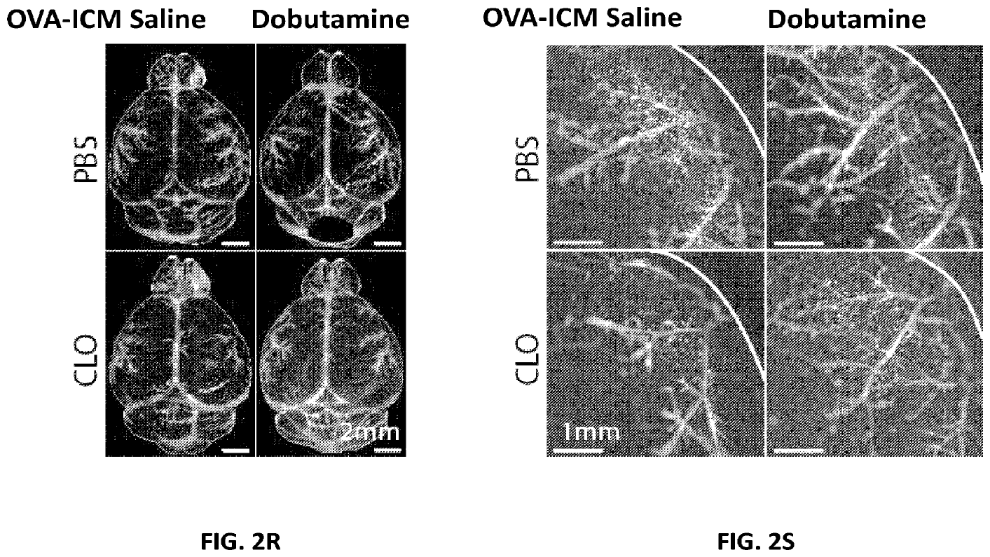

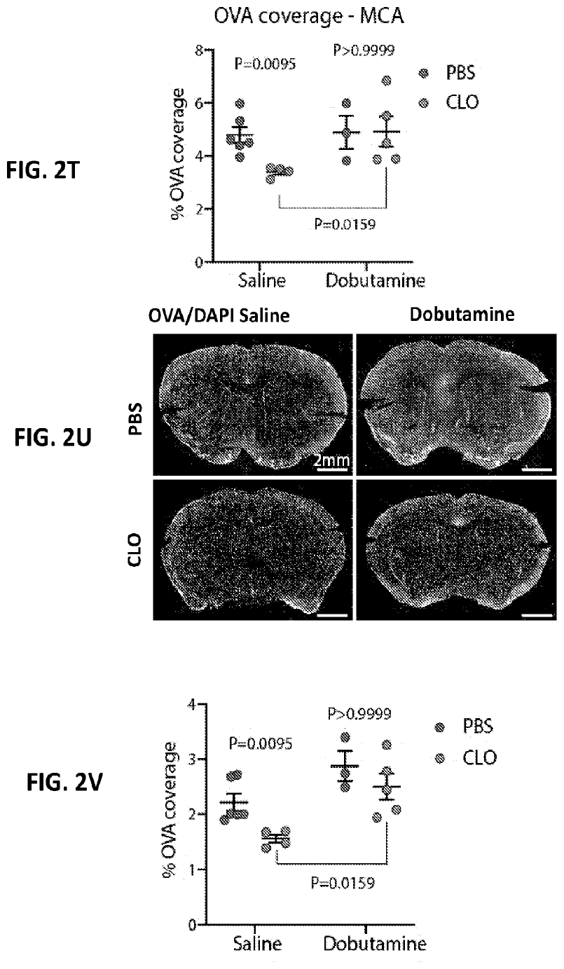

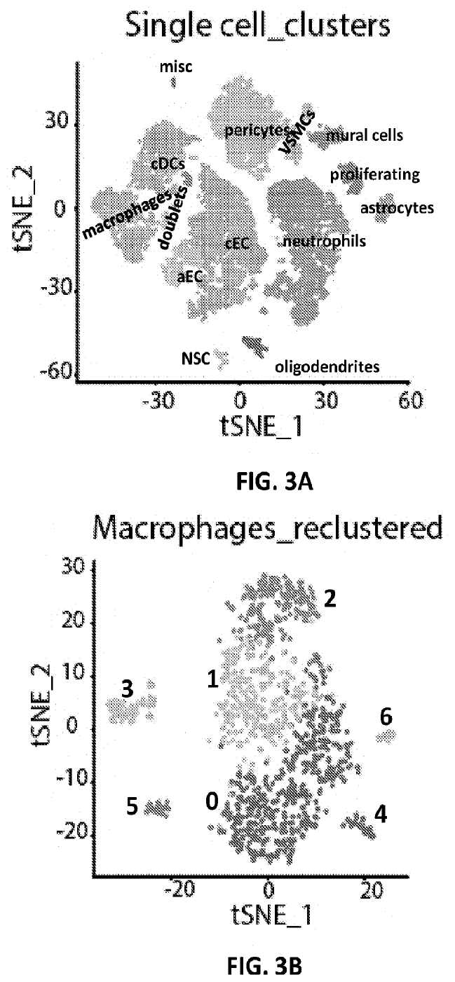

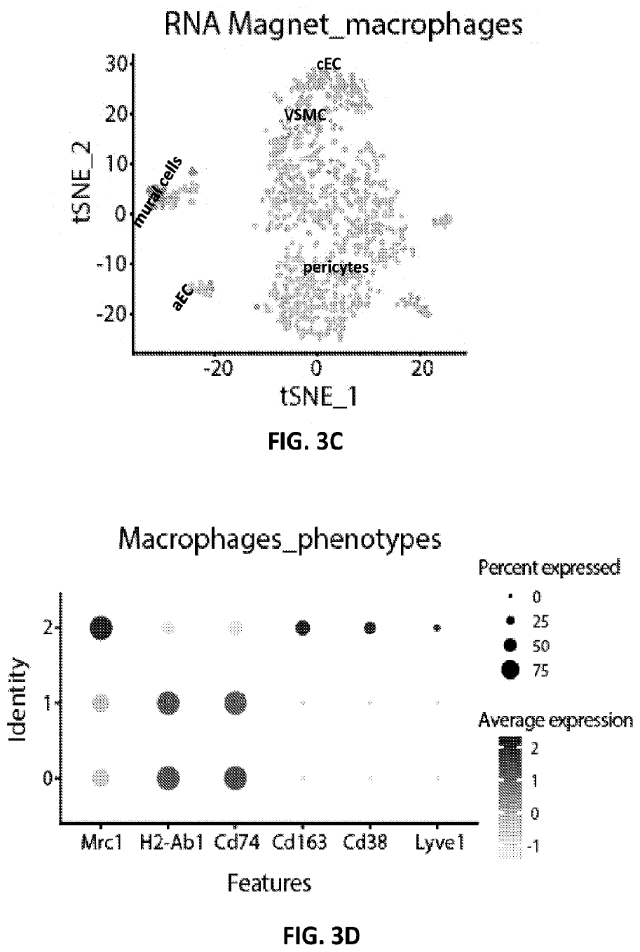

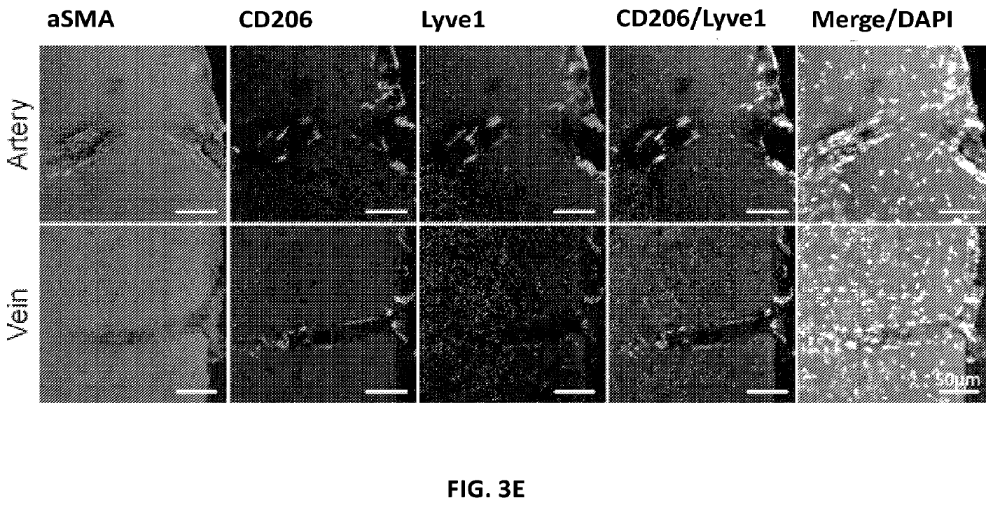

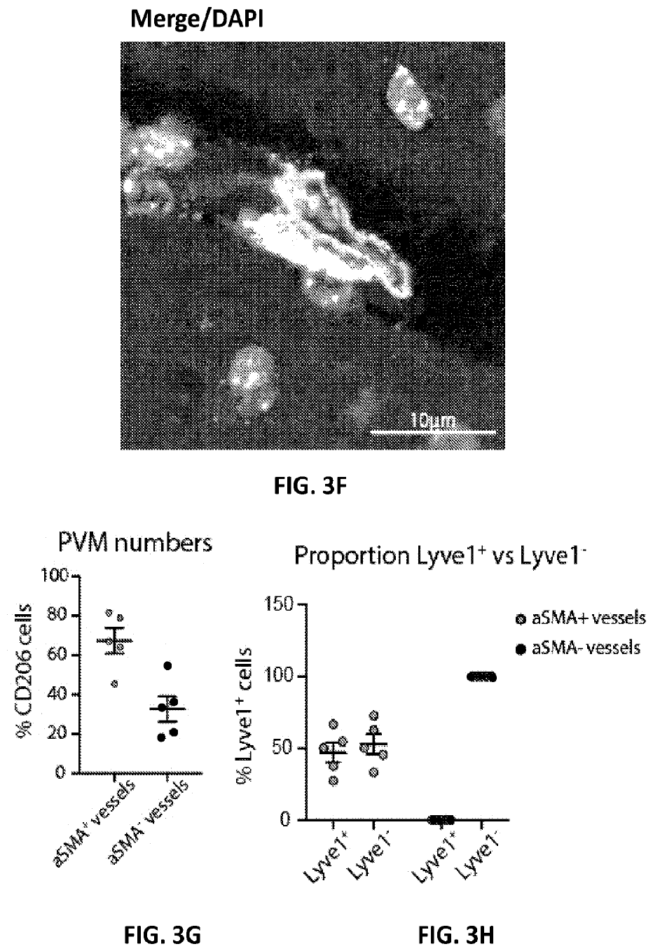

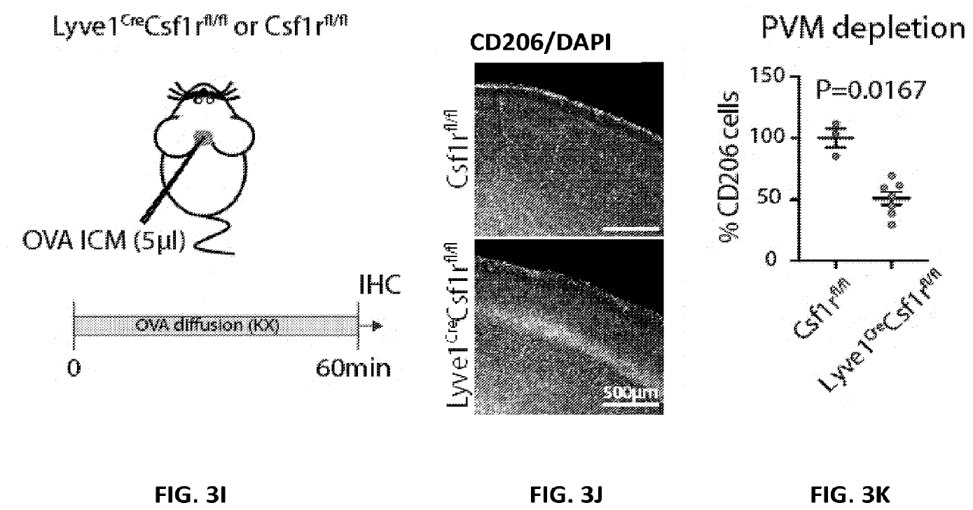

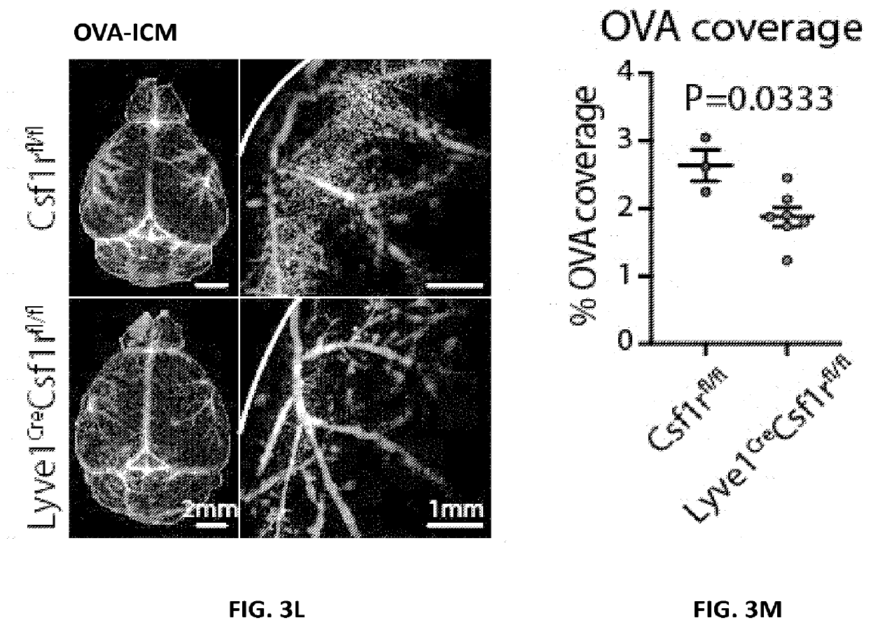

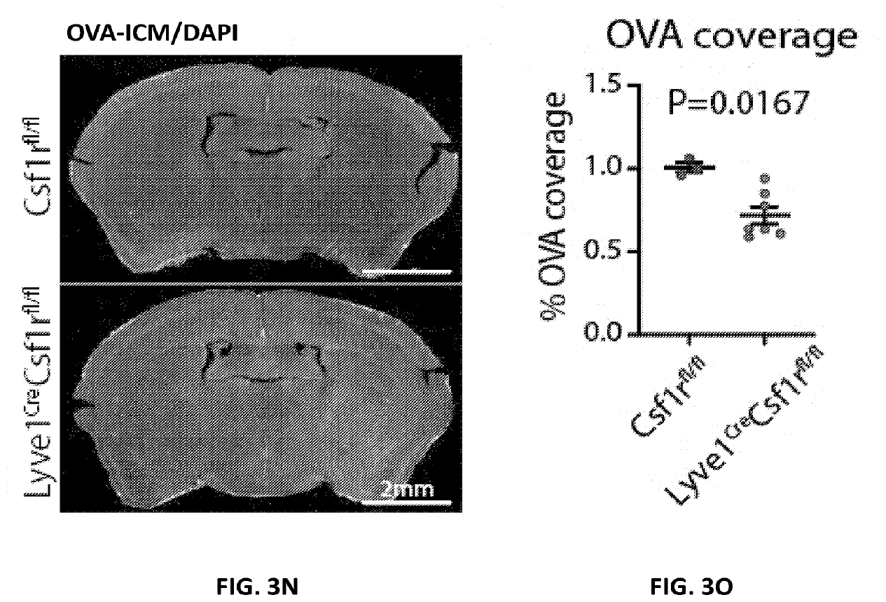

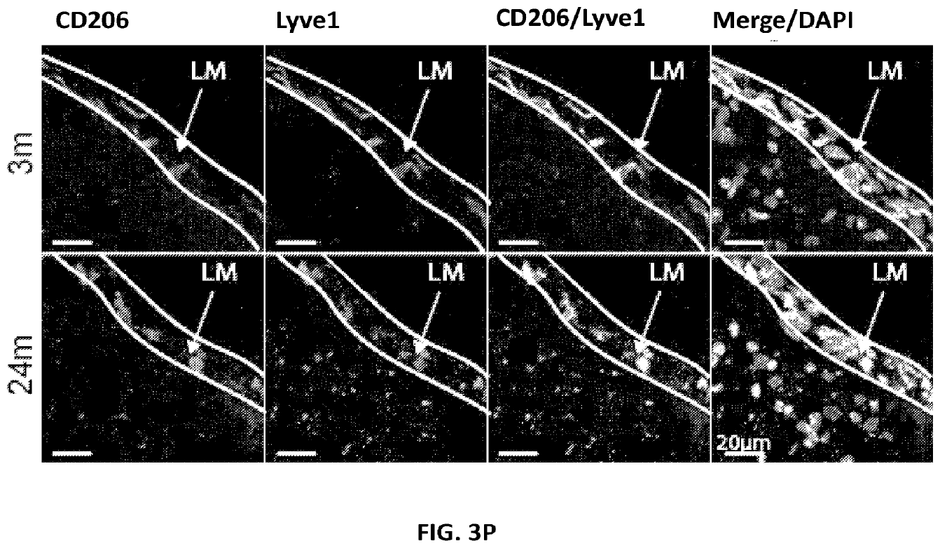

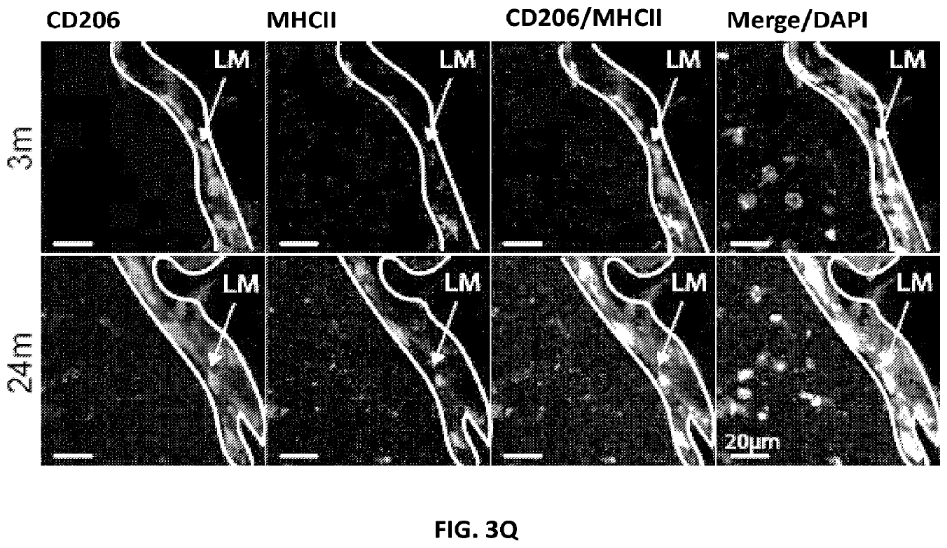

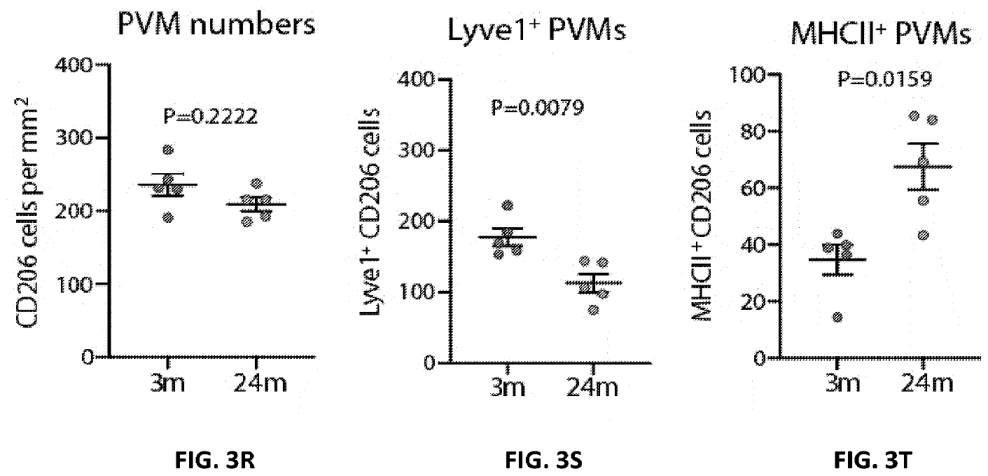

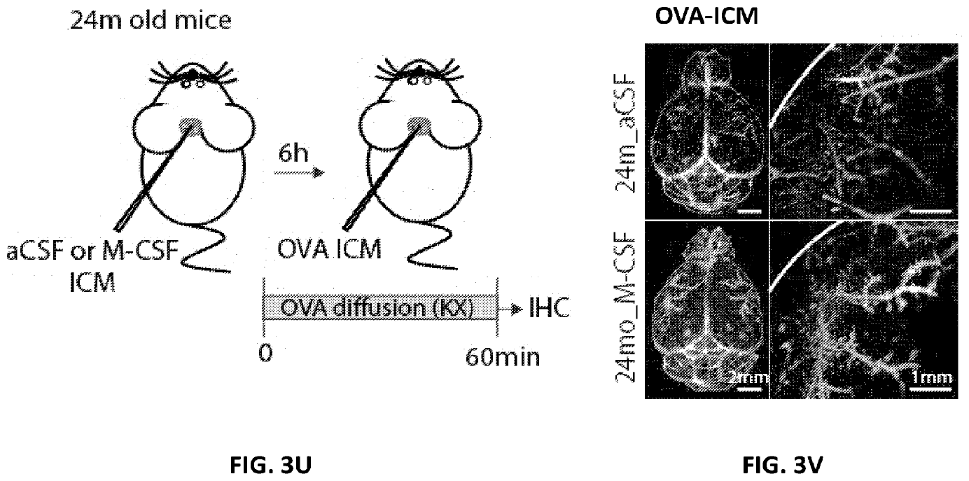

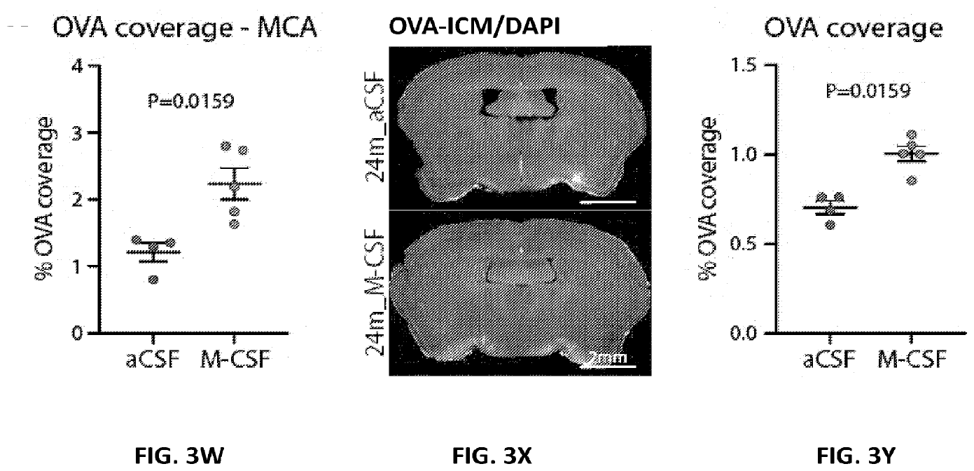

Those of skill in the art will understand that the drawings, described below, are for illustrative purposes only. The drawings are not intended to limit the scope of the present teachings in any way.

### DETAILED DESCRIPTION OF THE INVENTION

Brain border-associated macrophages are strategically located to protect the brain from pathogens, as well as sampling brain antigens. Perivascular macrophages (PVMs) are long-lived cells residing in the cerebrospinal fluid (CSF) within the perivascular space (PVS), a physical space delineated by the endothelium and the astrocytic endfeet. Without being limited to any particular theory, CSF is thought to diffuse from the PVS to the brain parenchyma. Within the brain parenchyma, the CSF may then mix with the local interstitial fluid and finally exit the brain, carrying along the brain's debris.

In various aspects, the compositions and methods disclosed herein are based on the discovery that perivascular macrophages (PVMs) act as mediators of brain perfusion by CSF. As illustrated in the examples herein, the health of cranial vasculature deteriorated without functional perivascular macrophages, resulting in poor perfusion of the CSF through the brain and accumulation of toxic compounds in the brain. The examples herein further demonstrated that impairing PVMs resulted in a worsening of Alzheimer's pathology in a mouse model. However, treating the mice with macrophage colony stimulating factor (M-CSF) via intracisternal magna (ICM) injection rejuvenated the perivascular macrophages and improved perfusion of the brains in older mice.

In various aspects, PVMs represent a new therapeutic target for age-associated diseases as well as diseases associated with an accumulation of debris in the brain, including, but not limited to, Alzheimer's Disease (AD), Parkinson's Disease (PD), and any other brain disease or disorder characterized by accumulation of debris within the brain.

### Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refers to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs the transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit the translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site, all other sequences of the gene and its controlling regions can be numbered. Downstream sequences (i.e., further protein-encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above required percent identities and retaining a required activity of the expressed protein are within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid which is replaced has a similar property as the original amino acid, for example, the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41 (fraction G/C content)−0.63(% formamide)−(600/l). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged | K R H |
| (Basic): | |
| Negatively Charged | D E |
| (Acidic): | |

Conservative Substitutions III

| Original | Exemplary |
|---|---|
| Residue | Substitution |
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, |
| | Phe, Ile, Val, Met, Ala, |
| Leu (L) | Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| | Ile, Leu, Met, Phe, |
| Val (V) | Ala |

Exemplary nucleic acids which may be introduced to a host cell include, for example, DNA sequences or genes from another species, or even genes or sequences which originate with or are present in the same species but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

### Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, intracisternal magna (ICM), subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for the treatment of the disease, disorder, or condition.

### Therapeutic Methods

Also provided is a process of treating, preventing, or reversing a brain disease or disorder characterized by accumulation of debris within the brain in a subject in need by administration of a therapeutically effective amount of a composition comprising a perivascular macrophage (PVM) stimulating compound, so as to prevent, reduce, or reverse the brain disease or disorder. Non-limiting examples of brain diseases or disorders characterized by accumulation of debris within the brain include Alzheimer's Disease (AD), Parkinson's Disease (PD), and any other brain disease or disorder characterized by accumulation of debris within the brain. Non-limiting examples of perivascular macrophage (PVM) stimulating compounds suitable for the therapeutic methods as disclosed herein include macrophage colony stimulating factor (M-CSF) and any other suitable perivascular macrophage (PVM) stimulating compound without limitation.

Methods described herein are generally performed on a subject in need thereof. A subject in need of the therapeutic methods described herein can be a subject having, diagnosed with, suspected of having, or at risk for developing a brain disease or disorder characterized by accumulation of debris within the brain. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of a perivascular macrophage (PVM) stimulating compound is, for example, an amount that would cause the desired therapeutic effect in a subject while minimizing undesired side effects. In various embodiments, an effective amount of perivascular macrophage (PVM) stimulating compound described herein can substantially inhibit a brain disease or disorder characterized by accumulation of debris within the brain, slow the progress of the brain disease or disorder, or limit the development of the brain disease or disorder.

According to the methods described herein, administration can be parenteral, pulmonary, oral, topical, intradermal, intramuscular, intraperitoneal, intravenous, intratumoral, intrathecal, intracranial, intracerebroventricular, intracisternal magna (ICM), subcutaneous, intranasal, epidural, ophthalmic, buccal, or rectal administration. In one aspect, the administration is intracisternal magna (ICM) injection.

When used in the treatments described herein, a therapeutically effective amount of perivascular macrophage (PVM) stimulating compound can be employed in pure form or, where such forms exist, in pharmaceutically form with or without a pharmaceutically acceptable excipient. For example, the compounds of the present disclosure can be administered, at a reasonable benefit/risk ratio applicable to any medical treatment, in a sufficient amount to prevent, reduce, or reverse a brain disease or disorder characterized by accumulation of debris within the brain.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

In some embodiments, the composition comprising the perivascular macrophage (PVM) stimulating compound may be administered in an amount ranging from about 1 mg/kg to about 100 mg/kg, from about 1 mg/kg to about 50 mg/kg, from about 1 mg/kg to about 25 mg/kg, from about 1 mg/kg to about 15 mg/kg, from about 1 mg/kg to about 10 mg/kg, from about 1 mg/kg to about 5 mg/kg, or from about 3 mg/kg to about 5 mg/kg. In some embodiments, the composition comprising the perivascular macrophage (PVM) stimulating compound described herein may be administered in an amount ranging from about 1 mg/kg to about 200 mg/kg, about 50 mg/kg to about 200 mg/kg, about 50 mg/kg to about 100 mg/kg, about 75 mg/kg to about 100 mg/kg, or about 100 mg/kg.

In some embodiments, the therapeutically effective amount of the composition comprising the perivascular macrophage (PVM) stimulating compound may be less than 1000 mg/kg/day, less than 500 mg/kg/day, less than 250 mg/kg/day, less than 100 mg/kg/day, less than 50 mg/kg/day, less than 25 mg/kg/day or less than 10 mg/kg/day. In other embodiments, the therapeutically effective amount of the composition may range from about 1 mg/kg/day to about 200 mg/kg/day.

In other non-limiting examples, a therapeutically effective amount of the composition comprising the perivascular macrophage (PVM) stimulating compound may range from about 1 microgram/kg/body weight, from about 5 microgram/kg/body weight, from about 10 microgram/kg/body weight, from about 50 microgram/kg/body weight, from about 100 microgram/kg/body weight, from about 200 microgram/kg/body weight, from about 350 microgram/kg/body weight, from about 500 microgram/kg/body weight, from about 1 milligram/kg/body weight, from about 5 milligram/kg/body weight, from about 10 milligram/kg/body weight, from about 50 milligram/kg/body weight, from about 100 milligram/kg/body weight, from about 200 milligram/kg/body weight, from about 350 milligram/kg/body weight, or from about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, or from about 5 microgram/kg/body weight to about 500 milligram/kg/body weight, etc., can be administered, based on the ranges described above.

Each of the states, diseases, disorders, and conditions, described herein, as well as others, can benefit from the compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of the perivascular macrophage (PVM) stimulating compound can occur as a single event or over a time course of treatment. For example, the perivascular macrophage (PVM) stimulating compound can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for prevention, reduction, or reversal of the brain disease or disorder characterized by accumulation of debris within the brain.

The perivascular macrophage (PVM) stimulating compound can be administered simultaneously or sequentially with another agent, such as an antibiotic, an anti-inflammatory, or another agent. For example, the perivascular macrophage (PVM) stimulating compound can be administered simultaneously with another agent, such as an antibiotic or an anti-inflammatory. Simultaneous administration can occur through the administration of separate compositions, each containing one or more of the perivascular macrophage (PVM) stimulating compound, an antibiotic, an anti-inflammatory, or another agent. Simultaneous administration can occur through the administration of one composition containing two or more of the perivascular macrophage (PVM) stimulating compound, an antibiotic, an anti-inflammatory, or another agent. The perivascular macrophage (PVM) stimulating compound can be administered sequentially with an antibiotic, an anti-inflammatory, or another agent. For example, the perivascular macrophage (PVM) stimulating compound can be administered before or after the administration of an antibiotic, an anti-inflammatory, or another agent.

### Administration

An aspect of the present disclosure provides for the perivascular macrophage (PVM) stimulating compound to be directly administered to a subject.

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, intracisternal magna (ICM), subcutaneous, intranasal, epidural, intrathecal, ophthalmic, transdermal, buccal, and rectal. In one aspect, the perivascular macrophage (PVM) stimulating compound may be directly administered to the subject by intracisternal magna (ICM) injection.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve the taste of the product; or improve the shelf life of the product.

### Screening

Also provided are methods for screening.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example: ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character x log P of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character x log P of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of compounds during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

### Kits

Also provided are kits. Such kits can include a perivascular macrophage (PVM) stimulating compound as described herein and, in certain embodiments, instructions for administration. Such kits can facilitate the performance of the methods described herein. When supplied as a kit, the different components of the composition can be packaged in separate containers and admixed immediately before use. Components include, but are not limited to compositions containing meningeal immune cells as described herein. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing the activity of the components.

Kits may also include reagents in separate containers such as, for example, sterile water or saline to be added to a lyophilized active component packaged separately. For example, sealed glass ampules may contain a lyophilized component and in a separate ampule, sterile water and sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet website specified by the manufacturer or distributor of the kit.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Perivascular Macrophages in Aging and Age-Associated Diseases

To characterize the role of perivascular macrophages (PVM) in the context of brain perfusion and brain diseases or disorders characterized by accumulation of debris within the brain, the following experiments were conducted.

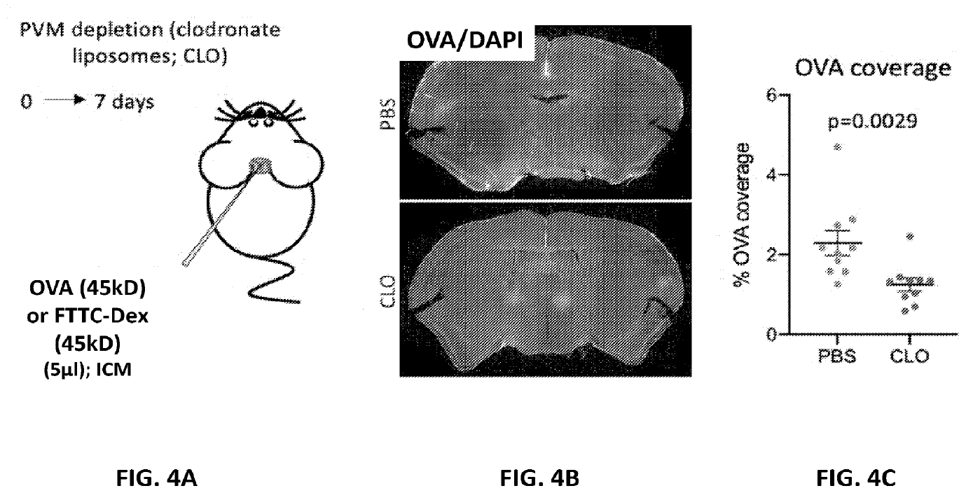

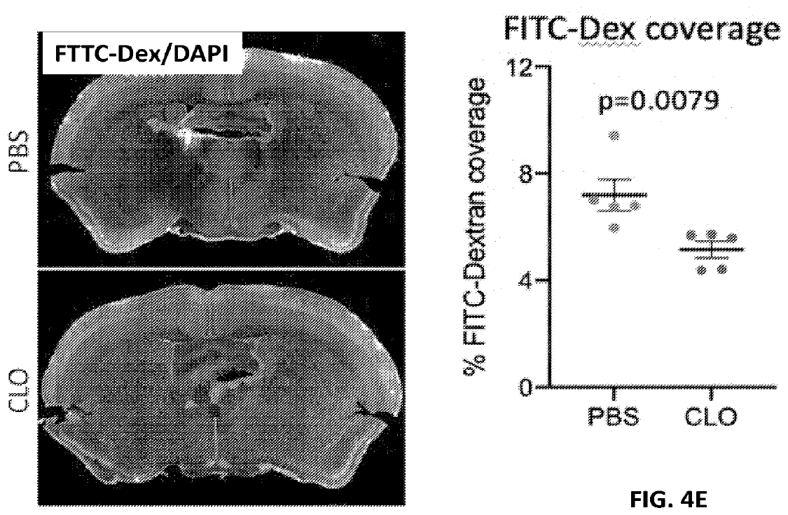

Brain border-associated macrophages are strategically located to protect the brain from pathogens, as well as sampling brain antigens. Perivascular macrophages (PVM) are long-lived cells residing in the cerebrospinal fluid (CSF) and more precisely in the perivascular space (PVS), a physical space delineated by the endothelium and the astrocytic endfeet. Recent studies demonstrated that the CSF could diffuse from the PVS to the brain parenchyma. The CSF will then mix with the local interstitial fluid and finally exit the brain, carrying along the brain's debris. In this example, the potential role of PVMs in CSF diffusion through the brain was investigated. One week after PVM depletion using clodronate liposomes, different fluorescent tracers were injected into the CSF to evaluate CSF diffusion. Interestingly, PVM depletion dramatically impaired CSF diffusion through the brain parenchyma (FIGS. 4A, 4B, 4C, 4D, and 4E). Furthermore, accumulation of tracers was observed when injected into the parenchyma itself, meaning that both influx and efflux of CSF are impaired after PVM depletion.

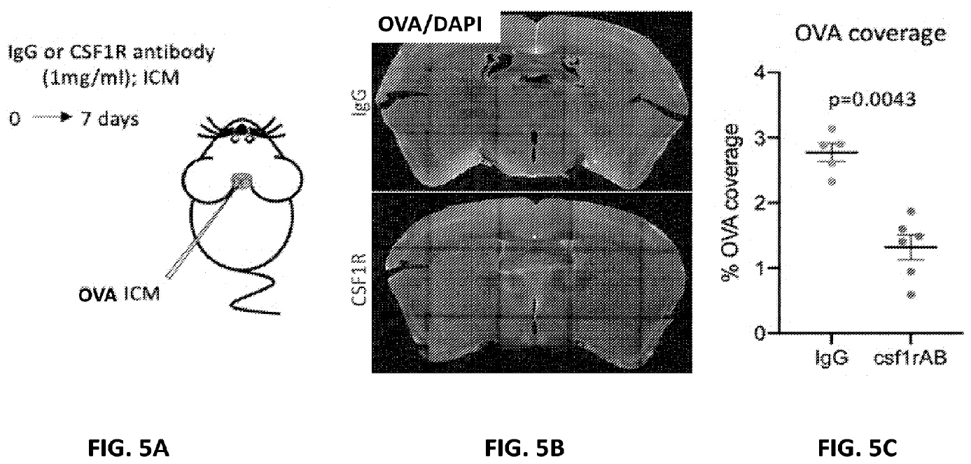

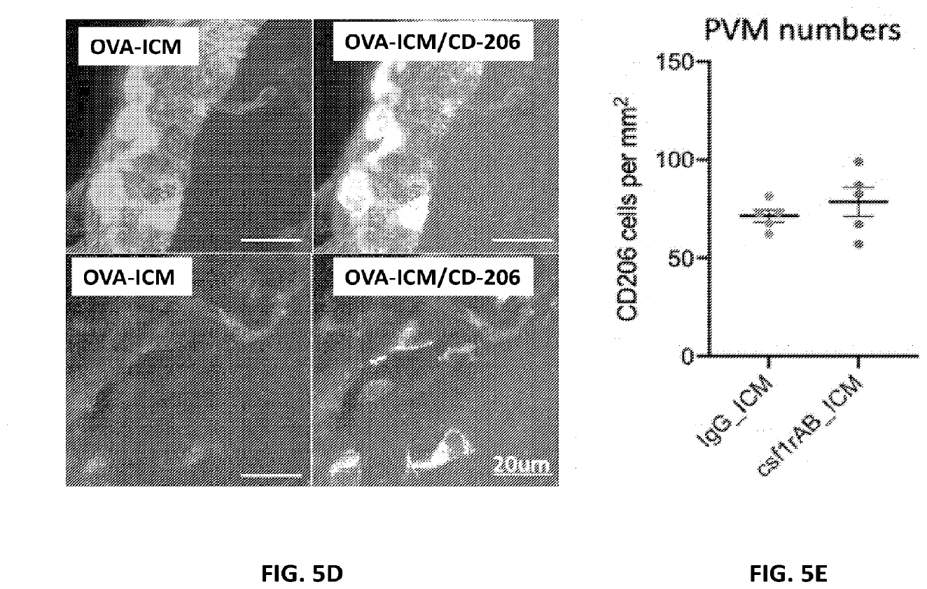

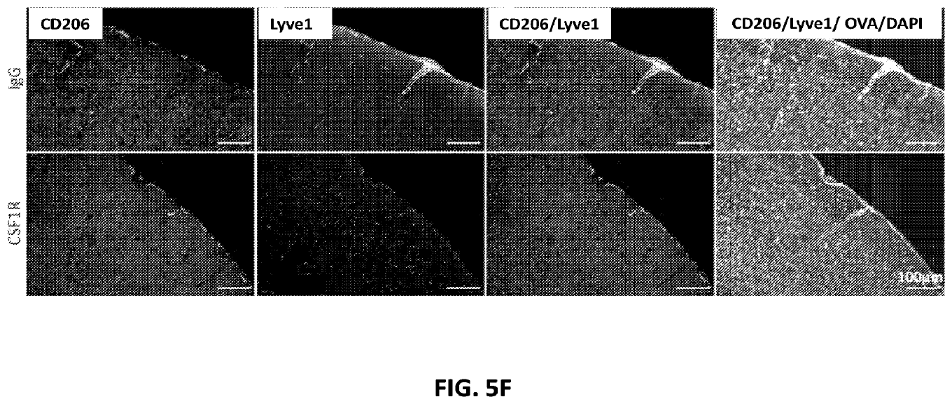

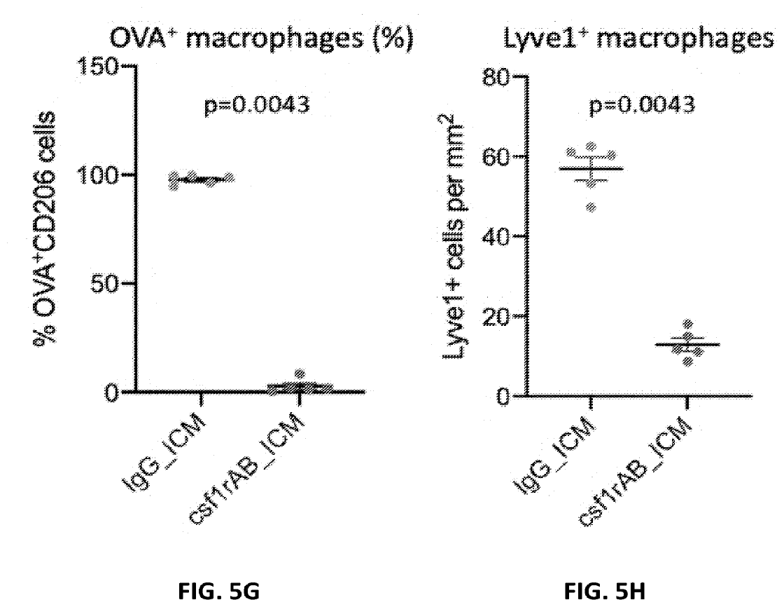

To confirm these results using another depletion approach, i.e.m. injection of neutralizing antibody targeting CSF1R was used. Even though PVMs were still present in CSF1R-treated mice, their phenotype was impaired—they were not able to phagocytose the tracers and were lacking scavenger markers such as Lyve1. Indeed, this impairment of PVM function was sufficient to result in reduced perfusion of the brain by the CSF (FIGS. 5A, 5B, 5C, 5D, 5E, 5F, 5G, and 5H).

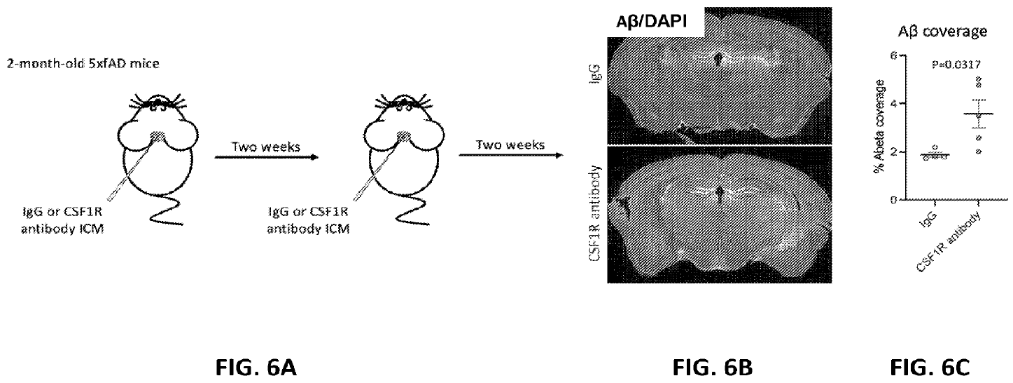

It was hypothesized that PVMs, through control of CSF diffusion, might affect plaque load. CSF1R antibodies were applied to 5×fAD mice, a mouse strain that overexpresses amyloid beta (AR). After a month and two antibody injections, brains were assessed for plaque load. It was found that CSF1R-treated 5×fAD mice have more plaques than IgG-treated 5×fAD mice (FIGS. 6A, 6B, and 6C).

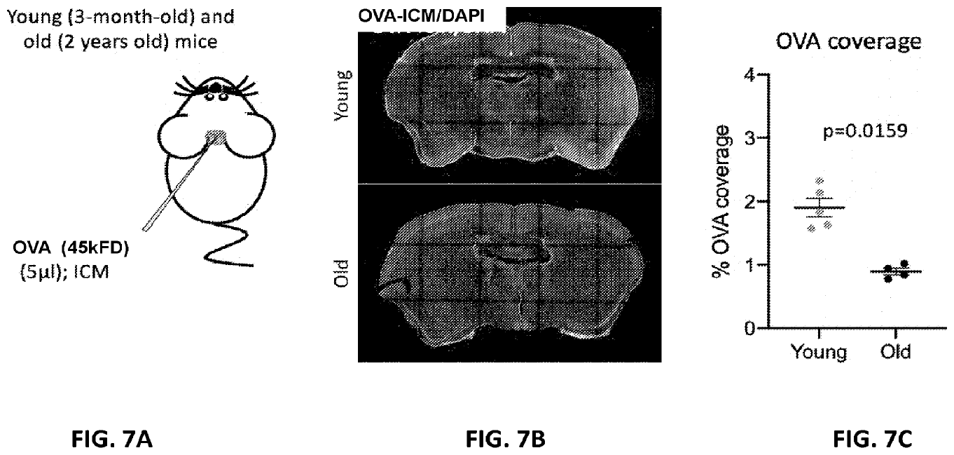

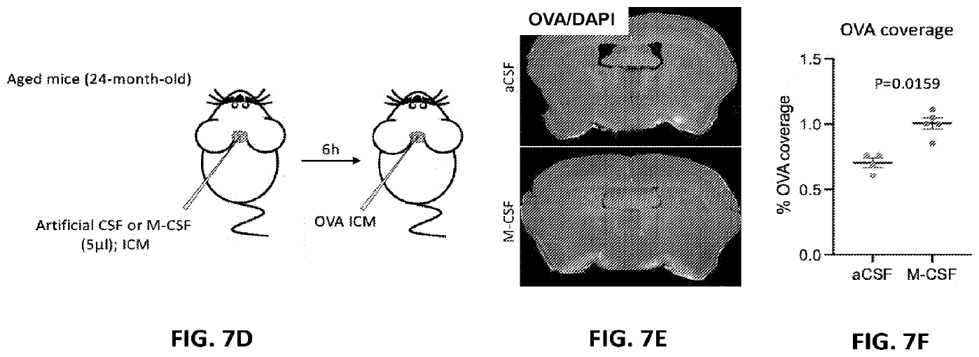

Finally, it was demonstrated that PVMs can be potential targets to rejuvenate CSF flow impairment in aged mice. First, it was observed that tracer diffusion in aged mice (24-month-old) was impaired compared to young/adult mice (3-month-old) (FIGS. 7A, 7B, 7C, 7D, 7E, and 7F). These results are in line with previously published data from our laboratory. Injection of M-CSF (or artificial CSF as control) i.e.m. resulted in improved tracer diffusion through the brain tissue (FIGS. 7E and 7F).

In conclusion, it was shown that PVMs had a modulating role in aging and age-associated diseases such as AD, through their control of CSF flow into the brain parenchyma, and could be therapeutically targeted in different neurological disorders.

What is claimed is:

1. A method of treating, preventing, or reversing a brain disease or disorder characterized by accumulation of debris within a brain of a subject, the method comprising administering a therapeutically effective amount of a composition comprising a perivascular macrophage (PVM) stimulating compound to the subject.

2. The method of claim 1, wherein the PVM stimulating compound comprises macrophage colony stimulating factor (M-CSF).

3. The method claim 1, wherein the brain disease or disorder characterized by accumulation of debris within the brain in a subject is selected from Alzheimer's Disease (AD) and Parkinson's Disease (PD).

4. The method of claim 1, wherein administering the therapeutically effective amount of the composition further comprises administering the composition by intracisternal magna (ICM) injection.
